(19) United States
(12) Patent Application Publication
(10) Pub. No.: US 2024/0188905 A1
(43) Pub. Date: Jun. 13, 2024

(54) AI-BASED LIVING SENSOR-LINKED CARE SERVICE PROVISION SYSTEM

(71) Applicant: NURION Co., Ltd., Ansan-si (KR)

(72) Inventors: Sang Woo LEE, Seoul (KR); Jeong Myung LEE, Gunpo-si (KR)

(73) Assignee: NURION Co., Ltd., Ansan-si (KR)

(21) Appl. No.: 18/538,352

(22) Filed: Dec. 13, 2023

(30) Foreign Application Priority Data
Dec. 13, 2022 (KR) 10-2022-0173753

(51) Int. Cl. A61B 5/00; A61B 5/0205; A61B 5/05; A61B 5/11; G10L 15/22

(52) U.S. Cl. CPC A61B 5/746; A61B 5/0022; A61B 5/02055; A61B 5/05; A61B 5/1126; A61B 5/741; A61B 5/749; G10L 15/22; A61B 5/02444

(57) ABSTRACT

An AI-based system for providing care services, includes a radar sensor installed in a specific space, obtains, in the specific space, life signals, and obtains information about the user's body temperature; an artificial intelligence speaker that is linked to the radar sensor, outputs a voice message to the user based on the information obtained by the radar sensor to determine whether an emergency has occurred to the user, and outputs a voice message to encourage the user to measure his/her body temperature at a specified time to allow the user to get into the habit of measuring his/her body temperature; a monitoring server that receives the life signals and analyzes the received life signals, senses an emergency that has occurred to the user and checks whether the emergency has actually occurred through the artificial intelligence speaker, and then provides an alarm about the emergency; and a user terminal.

### CROSS-REFERENCE TO PRIOR APPLICATION

This Application claims priority to Korean Patent Application No. 10-2022-0173753 (filed on Dec. 13, 2022), which is hereby incorporated by reference in its entirety.

### BACKGROUND

The present disclosure relates to an AI-based and life sensor-linked technology for providing care services, and, more particularly, to an AI-based and life sensor-linked system for providing care services for effectively sensing an emergency that has occurred to a user and quickly rescuing the user.

In general, a society where the proportion of the population aged 65 or older is seven percent or more is called an aging society, a society with 14 percent or more is called an aged society, and a society with 20 percent or more is called a post-aged society or a super-aged society.

In addition to the aging population, the number of single-person households has recently increased, so that the number of lonely deaths that are discovered after being left unattended for a long time has been increased. Accordingly, local governments at each level are making efforts to prevent lonely deaths by having social workers and volunteers regularly visit elderly people living alone or check their health by phone.

However, such a system has a problem in that, due to limited manpower, it is not possible to check the status of elderly people living alone in real time and respond quickly when an emergency situation occurs.

Accordingly, research and development has been actively conducted on a monitoring system that uses information and communication technology to install monitoring devices in the homes of seniors living alone and automatically contact guardians or emergency centers when an emergency situation occurs.

As a representative example, an artificial intelligence (AI) speaker hat can have simple conversations with users has been developed. An AI speaker refers to a speaker device that recognizes a user's voice, understands the content of the user's command, and then responds to the command. Such AI speakers are applied to various areas of life to improve the quality of life, and, in particular, are very useful as they have a function of providing care services for the elderly.

However, the conventional AI speakers have limitations in performing the function of providing care services for seniors because they are only capable of communicate with users simply answering their questions without recognizing their situations.

### PATENT LITERATURE

Korean Patent No. 10-2416469 (Jun. 29, 2022)
Korean Patent No. 10-2000644 (Jul. 10, 2019)

### SUMMARY

The present disclosure is aimed at providing an AI-based and life sensor-linked system for providing care services for effectively sensing an emergency that has occurred to a user and quickly rescuing the user.

The present disclosure is aimed at providing an AI-based and life sensor-linked system for providing care services that senses, through a radar sensor, a variety of life signals including information on whether a user is in the space, the user's movements, the user's breathing rate, the user's heart rate, the user's body temperature, whether the user has fallen, etc. to check the user's health and effectively sense the occurrence of an emergency.

The present disclosure is aimed at providing an AI-based and life sensor-linked system for providing care services that encourages a user to measure his/her bio-signals and make a habit of doing so and guides the user through actions the user can take based on the results of measuring the user's bio-signals by communicating with the user.

An AI-based system for providing care services according to an embodiment of the present disclosure may include a radar sensor that is installed in a specific space, obtains, in the specific space, life signals including information on whether the user is in the space, the user's movements, the user's breathing, and the user's heart rate without being in contact with a user, and obtains information about the user's body temperature among the life signals by being in contact with the user; an artificial intelligence speaker that is linked to the radar sensor, outputs a voice message to the user based on the information obtained by the radar sensor to determine whether an emergency has occurred to the user, and outputs a voice message to encourage the user to measure his/her body temperature at a specified time to allow the user to get into the habit of measuring his/her body temperature; a monitoring server that receives the life signals collected by the radar sensor and analyzes the received life signals to monitor the specific space, senses an emergency that has occurred to the user and checks whether the emergency has actually occurred through the artificial intelligence speaker, and then provides an alarm about the emergency; and a user terminal linked to each of the radar sensor, the artificial intelligence speaker, and the monitoring server through a dedicated application.

The radar sensor may include a life sensing module that transmits a radar signal into a sensor sensing area formed in front and then receives a reflected signal to measure the life signals; a body temperature measurement module that includes a body temperature sensor and measures the user's body temperature when in contact with the user through the body temperature sensor; and a communication module that is connected, through a network, with the artificial intelligence speaker, the monitoring server, or the user terminal to provide a communication function.

The artificial intelligence speaker may include a voice recognition module that determines whether an emergency has occurred by recognizing the user's voice; an action alarm module that automatically outputs a voice message to encourage the user to measure his/her body temperature at a specified time, analyzes the user's voice response to determine whether the user wants to measure his/her body temperature, and outputs voice messages to encourage the user to take the steps to measure his/her body temperature according to the order of measuring body temperature when the user wants to measure his/her body temperature; and a communication module that is connected, through a network, with the radar sensor, the monitoring server, or the user terminal to provide a communication function.

The artificial intelligence speaker may receive information collected by the radar sensor from the voice recognition module to carry out voice commands and generate an event signal when an obtained voice command corresponds to an emergency call.

When the user's voice message to proceed a self-measurement of his/her body temperature is sensed before the action alarm module automatically outputs a voice message to encourage the user to measure his/her body temperature at a specified time, the artificial intelligence speaker may prevent the voice message to encourage the user to measure his/her body temperature from being automatically output.

When the event signal is received, the monitoring server may send an alarm about the user's emergency call to the user terminal of a pre-designated manager or guardian.

When the occurrence of an event resulting from the emergency call is sensed, the monitoring server may provide biometric information about the user's breathing, the user's heart rate, and the user's body temperature along with an alarm about the occurrence of the event to an external designated organization.

The monitoring server may determine whether the user is in the space based on the life signals, monitor whether the user is moving based on whether the user is in the space, classify the ranges of body temperature into unmeasurable, to be remeasured, normal, normal (slight fever), abnormal (high fever), and abnormal (high temperature and fire) and set an alarm for each range, and send an alarm to the user terminal of a pre-designated manger or guardian when the value of the measured body temperature falls within the range of abnormal (high fever) and allow the artificial intelligence speaker to output a voice message to re-measure the user's body temperature after a certain period of time.

The present disclosure may have the following effects. However, because it does not mean that a specific embodiment must include all of the following effects or only the following effects, the claims of the present disclosure should not be deemed to be limited thereto.

According to an embodiment of the present disclosure, it may be possible for the AI-based and life sensor-linked system for providing care services to effectively sense an emergency that has occurred to a user and quickly rescue the user.

According to an embodiment of the present disclosure, it may be possible for the AI-based and life sensor-linked system for providing care services to sense, through a radar sensor, a variety of life signals including information on whether a user is in the space, the user's movements, the user's breathing rate, the user's heart rate, the user's body temperature, whether the user has fallen, etc. in order to check the user's health and effectively sense the occurrence of an emergency.

According to an embodiment of the present disclosure, it may be possible for the AI-based and life sensor-linked system for providing care services to encourage a user to measure his/her bio-signals and make a habit of doing so and to guide the user through actions the user can take based on the results of measuring the user's bio-signals, by communicating with the user.

### BRIEF DESCRIPTION OF THE DRAWINGS

FIG. 1 is a view for illustrating an AI-based and life sensor-linked system for providing care services according to the present disclosure.

FIG. 2 is a view for illustrating the features of the radar sensor in FIG. 1.

FIG. 3 is a view for illustrating the features of the artificial intelligence speaker in FIG. 1.

FIG. 4 is a view for illustrating the features of the monitoring server in FIG. 1.

FIG. 5 is a flowchart for illustrating an embodiment of the operation of the system for providing care services according to the present disclosure.

FIG. 6 is a flowchart for illustrating the process of measuring body temperature based on voice messages automatically output by the artificial intelligence speaker of the system for providing care services according to the present disclosure.

FIG. 7 is a flowchart for illustrating the process of measuring body temperature based on a user's voice messages by the system for providing care services according to the present disclosure.

### DETAILED DESCRIPTION

A description of the present disclosure is merely an embodiment for a structural or functional description and the scope of the present disclosure should not be construed as being limited by an embodiment described in a text. That is, since the embodiment can be variously changed and have various forms, the scope of the present disclosure should be understood to include equivalents capable of realizing the technical spirit. Further, it should be understood that since a specific embodiment should include all objects or effects or include only the effect, the scope of the present disclosure is limited by the object or effect.

Meanwhile, meanings of terms described in the present application should be understood as follows.

The terms “first,” “second,” and the like are used to differentiate a certain component from other components, but the scope of should not be construed to be limited by the terms. For example, a first component may be referred to as a second component, and similarly, the second component may be referred to as the first component.

It should be understood that, when it is described that a component is “connected to” another component, the component may be directly connected to another component or a third component may be present therebetween. In contrast, it should be understood that, when it is described that an element is “directly connected to” another element, it is understood that no element is present between the element and another element. Meanwhile, other expressions describing the relationship of the components, that is, expressions such as “between” and “directly between” or “adjacent to” and “directly adjacent to” should be similarly interpreted.

It is to be understood that the singular expression encompasses a plurality of expressions unless the context clearly dictates otherwise and it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

In each step, reference numerals (e.g., a, b, c, etc.) are used for convenience of description, the reference numerals are not used to describe the order of the steps and unless otherwise stated, it may occur differently from the order specified. That is, the respective steps may be performed similarly to the specified order, performed substantially simultaneously, and performed in an opposite order.

The present disclosure can be implemented as a computer-readable code on a computer-readable recording medium and the computer-readable recording medium includes all types of recording devices for storing data that can be read by a computer system. Examples of the computer readable recording medium may include a ROM, a RAM, a CD-ROM, a magnetic tape, a floppy disk, an optical data storage device, and the like. Further, the computer readable recording media may be stored and executed as codes which may be distributed in the computer system connected through a network and read by a computer in a distribution method.

If it is not contrarily defined, all terms used herein have the same meanings as those generally understood by those skilled in the art. Terms which are defined in a generally used dictionary should be interpreted to have the same meanings as the meanings in the context of the related art, and are not interpreted as ideal meanings or excessively formal meanings unless clearly defined in the present application.

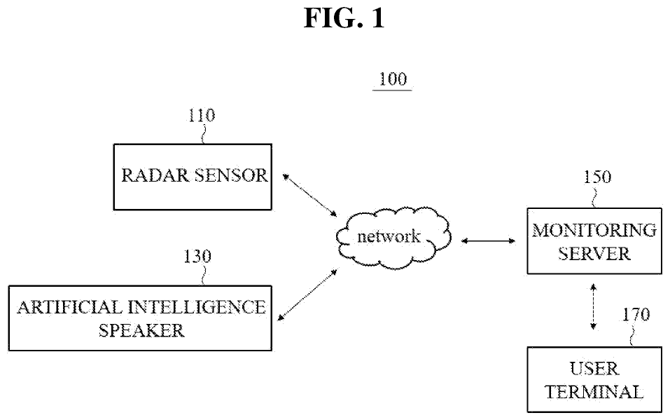

FIG. 1 is a view for illustrating an AI-based and life sensor-linked system for providing care services according to the present disclosure.

Referring to FIG. 1, a system 100 for providing care services may include a radar sensor 110, an artificial intelligence speaker 130, a monitoring server 150, and a user terminal 170.

The radar sensor 110 may be installed in a specific place and collect information about life reactions (i.e., life signals) occurring in the place. To this end, the radar sensor 110 may use a Doppler radar to collect biometric information about whether a user is in the space, whether the user's movements have been sensed, whether the user has fallen, the user's respiratory rate, the user's heart rate, the user's body temperature, etc. More specifically, the radar sensor 110 may extract information about various characteristics such as signal strength, change amount, and change pattern from a received signal and determine a range of information about the situation to monitor the situation based on the extracted information. For example, the information about the situation may include information about whether a user is in a specific space, whether the user is moving in that specific space, and whether the user has fallen from the bed. The information about the situation may include information about the user's breathing rate and heart rate derived from biometric signals. In addition, the radar sensor 110 may include a body temperature sensor, measure a user's body temperature through the body temperature sensor, and include information about the user's body temperature, which has been measured, in information about the situation. Here, the radar sensor 110 may measure the user's body temperature upon the request for measuring the user's body temperature by an automatic voice command of the artificial intelligence speaker 130 or the user's voice command, and may provide voice guidance of the results of measuring the user's body temperature through the artificial intelligence speaker 130.

The radar sensor 110 may be connected to each of the artificial intelligence speaker 130, the monitoring server 150, and the user terminal 170 through a network, and, if necessary, a plurality of radar sensors 110 may be installed and operated in one indoor space. According to an embodiment of the present disclosure, the radar sensor 110 may be connected to the monitoring server 150 through a network via a gateway as needed.

In addition, the radar sensor 110 may include an antenna and a receiver for transmitting and receiving wireless signals, an amplifier for amplifying signals, a signal processor for processing signals, etc. The radar sensor 110 may use various wireless signals to measure biological signals, and may specify a frequency band as needed and perform an operation of measuring biological signals using only a certain frequency.

According to an embodiment of the present disclosure, the radar sensor 110 may transmit biological signals to the monitoring server 150 based on a predefined communication protocol. Here, the communication protocol may be defined as a biometric radar protocol. The type of data transmitted based on the biometric radar protocol may indicate MAC address, WiFi RSSI, whether a user is in the space, the user's movements, the user's respiratory rate, the user's heart rate, peak-to-peak (P to P), sensitivity, a reference value, a management number, a service set identifier (SSID), the user's body temperature, and a body temperature alarm. Here, the value of the body temperature may be transmitted every five seconds and displayed to one decimal place. For example, a body temperature of 35.0 degrees is displayed as “350.” The body temperature alarm may refer to information about the user's condition for each range of body temperature and may be displayed as 0 to 5. Here, the body temperature alarm may be defined as shown in Table 1 below (“N/A” indicates that body temperature cannot be measured).

TABLE 1

| | | Body Temperature | |
|---|---|---|---|
| Body Temperature | | Alarm | Condition |
| ~33.0° | C. | 0 | N/A |
| 33.1° C.~35.0° C. | | 1 | Normal |
| 35.1° C.~37.8° C. | | 2 | Normal |
| 37.9° C.~39.5° C. | | 3 | Normal (Mild Fever) |
| 39.6° C.~42.0° C. | | 4 | Abnormal (High Fever) |
| 42.1° C.~55.0° C. | | 0 | N/A |
| 55.1° C.~85.0° C. | | 5 | Abnormal (High |
| | | | Temperature and Fire) |
| 85.1° | C.~ | 0 | N/A |

The artificial intelligence speaker 130 may recognize a user's time-series situation and automatically output a voice message appropriate for the situation in order to interact with the user and encourage the user to perform a necessary action. Here, the artificial intelligence speaker 130 may be provided integrally with the radar sensor 110, or may be provided as a separate device and connected wirelessly to the radar sensor 110.

According to an embodiment of the present disclosure, the artificial intelligence speaker 130 may determine the content of a voice message to encourage a user to get into the habit of measuring his/her body temperature and automatically output the voice message at a designated time to encourage the user to measure his/her body temperature. For example, the artificial intelligence speaker 130 may automatically output the voice message at 10 o'clock every day to encourage the user to measure his/her body temperature and determine whether the user has accepted it based on the user's voice response. In addition, the artificial intelligence speaker 130 may provide voice guidance on the results of measuring the user's body temperature by being linked to the radar sensor 110.

Furthermore, when an emergency situation is sensed by the radar sensor 110, the artificial intelligence speaker 130 linked to the radar sensor 110 may automatically output a voice message to ask a user whether an emergency has actually occurred. The artificial intelligence speaker 130 may determine the user's response by recognizing the user's voice. Here, when voice information indicating an emergency situation is recognized, the artificial intelligence speaker 130 may transmit it to the real-time monitoring server 150 to make an emergency call.

The monitoring server 150 may analyze bio-signals collected in a specific place to determine whether an emergency has occurred to a person living in that place (e.g., the elderly, a patient, etc.), and may be a server corresponding to a computer or a program that sends an alarm about a dangerous situation to the outside and remotely controls measures for emergency rescue. That is, the monitoring server 150 may generate information on apnea, cardiac arrest, breathing, falls, the quality of sleep, activity level, etc. as a result of analyzing the collected information, thereby conducting 24-hour monitoring.

The monitoring server 150 may be linked to a gateway through Ethernet, etc., and may manage information collected by the gateway by storing it in a database (DBMS).

Meanwhile, the monitoring server 150 may operate by being linked to an external system. For example, in order to quickly respond to an emergency occurring to a user or a user's voice requesting an emergency call, if any, the monitoring server 150 may be linked to hospitals, the 119 service, the police, etc. necessary for emergency rescue, and may be linked to a public system such as a community center to provide related information thereto.

The user terminal 170 may correspond to a computing device capable of receiving various information obtained by monitoring and an alarm about the occurrence of an emergency through the system 100 for providing care services. The user terminal 170 may be a smartphone, a laptop computer, or a desktop computer that can be operated by being connected to the monitoring server 150. However, the user terminal 170 is not necessarily limited thereto, and may be in the form of a variety of devices such as a tablet PC as well.

In addition, the user terminal 170 may install and execute a dedicated program or an application for being linked to the monitoring server 150. As a result, it may be possible for the user terminal 170 to use various services provided by the monitoring server 150.

Furthermore, the user terminal 170 may set parameters for being linked to the radar sensor 110 through a WiFi module-specific application. For example, the user terminal 170 may set the IP of a remote server, a WiFi router, an access SSID, a password, etc.

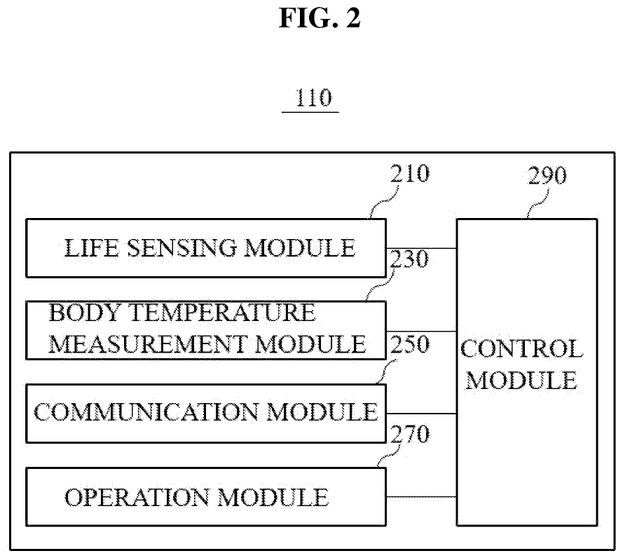

FIG. 2 is a view for illustrating the features of the radar sensor in FIG. 1.

Referring to FIG. 2, the radar sensor 110 may include a life sensing module 210, a body temperature measurement module 230, a communication module 250, an operation module 270, and a control module 290.

The life sensing module 210 may perform an operation of sensing an object based on a radar signal and measure signals related to a person's breathing, heart rate, etc. The life sensing module 210 may measure biological signals as a result of transmitting a radar signal into a sensor sensing area formed in front and then receiving a reflected signal. According to an embodiment of the present disclosure, the life sensing module 210 may radiate millimeter radio waves to the human body and extract bio-signals from reflected signals. Here, the life sensing module 210 may use 24 GHz micro Doppler, which is harmless to the human body. The life sensing module 210 may serve to collect life signals in real time or periodically.

The body temperature measurement module 230 may include a body temperature sensor, and may measure a user's body temperature by being in contact with the user and transmit the value of the measured body temperature to the artificial intelligence speaker 130 and the monitoring server 150.

The communication module 250 may connect the radar sensor 110 to the artificial intelligence speaker 130, the monitoring server 150, or the user terminal 170 through a network. The communication module 250 may be designed to facilitate a wireless communication system used by the radar sensor 110.

The operation module 270 may correspond to a calculation unit for performing the unique functions of the radar sensor 110. That is, while the control module 290 may serve to handle the linkage and the control between the operations of the radar sensor 110, the operation module 270 may serve to process the unique functions of the radar sensor 110. Information generated by the operation module 270 may be transmitted to the control module 290, and may be stored and managed in an internal storage space.

The control module 290 may control the overall operation of the radar sensor 110, and may manage control flow or data flow between the life sensing module 210, the body temperature measurement module 230, the communication module 250, and the operation module 270.

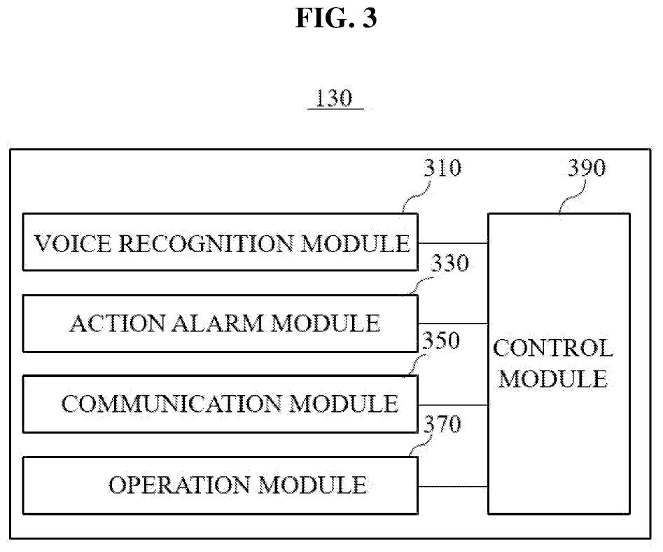

FIG. 3 is a view for illustrating the features of the artificial intelligence speaker in FIG. 1.

Referring to FIG. 3, the artificial intelligence speaker 130 may include a voice recognition module 310, an action alarm module 330, a communication module 350, an operation module 370, and a control module 390.

The voice recognition module 310 may communicate with a user, recognize the user's voice requesting help, and generate an emergency signal. According to an embodiment of the present disclosure, the voice recognition module 310 may predefine at least one command for an emergency call and then obtain a voice command by processing a user's voice signal into natural language, and may immediately generate an emergency call event when the voice command corresponds to the command for an emergency call. Here, the commands for an emergency call may include specific words requesting help, such as “save” and “help.”

According to an embodiment of the present disclosure, the voice recognition module 310 may perform analysis and processing to convert a user's voice, which has been recognized, into text (Speech to Text) and extract meaningful information by text mining. Here, the voice recognition module 310 may extract words or morphemes from the text through a pre-trained data recognition module. For example, the voice recognition module 310 may extract words or morphemes such as “me” and “help” from the text “help me” through a pre-trained data recognition model based on artificial intelligence (AI). When extracted words or morphemes are included in predefined commands for an emergency call, the voice recognition module 310 may output a signal for an emergency call to raise an alarm about an emergency occurring to a user. The signal for an emergency call may be transmitted to the monitoring server 150 and the user terminal 170 of a guardian/manager through a network so that information on whether the user is in an emergency condition may be provided.

The action alarm module 330 may determine the content of a voice message to encourage a user to take a specific action and output it at a specified time, and then determine whether the user will take the specific action based on the user's voice response. According to an embodiment of the present disclosure, the action alarm module 330 may automatically output a voice message to encourage a user to measure his/her body temperature at a specified time to allow the user to make a habit of measuring his/her body temperature. The action alarm module 300 may determine whether the user will measure his/her body temperature by analyzing the user's voice response to the voice message to encourage the user to measure his/her body temperature. When the user has decided to measure his/her body temperature, the action alarm module 330 may provide the user with a voice guidance on the procedures for measuring body temperature to enable the user to measure his/her body temperature. When a user makes a request for measuring his/her body temperature through the voice recognition module 310 before the action alarm module 330 automatically outputs a voice message to encourage the user to measure his/her body temperature, the action alarm module 330 may not output the voice message.

The communication module 350 may connect the artificial intelligence speaker 130 with the radar sensor 110, the monitoring server 150, or the user terminal 170 through a network. The communication module 350 may be designed to facilitate a communication system used by the artificial intelligence speaker 130.

The operation module 370 may correspond to a calculation unit for performing the unique functions of the artificial intelligence speaker 130. That is, while the control module 390 may serve to handle the linkage and the control between the operations of the artificial intelligence speaker 130, the operation module 370 may serve to process the unique functions of the artificial intelligence speaker 130. Information generated by the operation module 370 may be transmitted to the control module 390, and may be stored and managed in an internal storage space.

The control module 390 may control the overall operation of the artificial intelligence speaker 130, and may manage control flow or data flow between the voice recognition module 310, the action alarm module 330, the communication module 350, and the operation module 370.

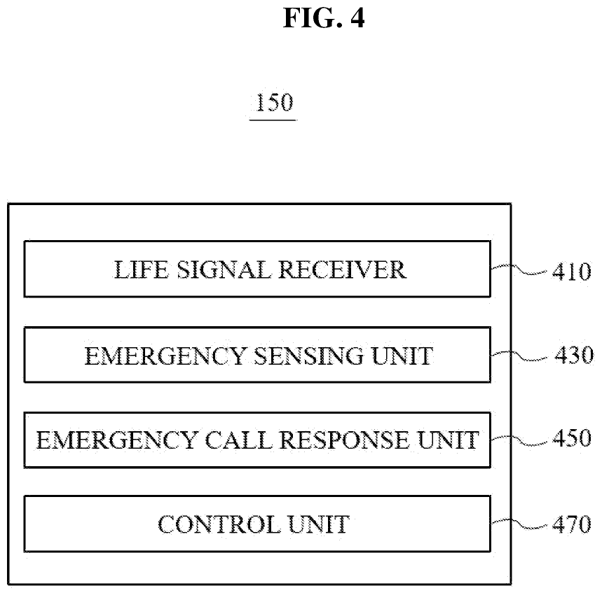

FIG. 4 is a view for illustrating the features of the monitoring server in FIG. 1.

Referring to FIG. 4, the monitoring server 130 may include a life signal receiver 410, an emergency sensing unit 430, an emergency call response unit 450, and a control unit 470.

The life signal receiver 410 may receive life signals as sensed information from the radar sensor 110. The life signal receiver 410 may classify and manage the received life signals by type. For example, the life signal receiver 410 may classify life signals in relation to whether a user is in the space, whether the user is moving, the user's respiratory rate, the user's heart rate, and the analysis of the user's body temperature. In addition, when an emergency call event signal is received from the radar sensor 110, the life signal receiver 410 may transmit the emergency call event signal to the emergency call response unit 450, so that immediate action can be taken for the emergency call.

According to an embodiment of the present disclosure, the life signal receiver 410 may provide various statistical information about life signals. The life signal receiver 410 may visualize and display the various statistical information derived from life signals, and may provide a graphical user interface (GUI) for this purpose.

The emergency sensing unit 430 may sense the occurrence of an emergency situation while monitoring a specific space by analyzing received life signals. When it is determined that an emergency has occurred to a user, the emergency sensing unit 430 may operate according to an operating scenario to raise an alarm for the emergency and respond thereto. For example, the operating scenario may be defined based on whether the user is in the space, the user's breathing, the user's heart rate, and the user's body temperature, and may be applied in the process of raising an alarm for the emergency and responding thereto.

According to an embodiment of the present disclosure, the emergency sensing unit 430 may determine whether a user is in the space based on life signals, monitor whether the user is carrying out daily activities when it is determined that the user is in the space, determine that the user is carrying out daily activities when movement data based on the user's movements are continuously generated for a predetermined first period of time, and may calculate and grade the activity level of the user's movements based on how many times it was determined that the user was carrying out routine activities for a predetermined second period of time.

More specifically, the emergency sensing unit 430 may determine whether a user is within the radar range, that is, whether the user is in the space, based on life signals. When the user is in a specific space, it may be divided into a first state where the user's movements are sensed and a second state where the user's movements are not sensed. The emergency sensing unit 430 may monitor whether the user's movements are sensed when the user is in the space.

In other words, the emergency sensing unit 430 may determine that a user is carrying out daily activities when movement data based on the user's movements are continuously generated for the predetermined first period of time, and may calculate and grade the activity level of the user's movements based on how many times it was determined that the user was carrying out routine activities for the predetermined second period of time. For example, it may be determined that the user is engaging in routine activities when the movement data are generated for three seconds or more, and the determination that the user is carrying out daily activities may be withdrawn when the user's movements are not sensed for 10 seconds or more. In addition, the activity level of the user's movements may be graded as high, medium, and low based on how many times it was determined that the user was carrying out routine activities for one hour.

According to an embodiment of the present disclosure, when it continues to fail to determine that a user is carrying out routine activities for a predetermined third period of time, the emergency sensing unit 430 may renew the determination on whether the user is in the space based on the user's respiratory rate and heart rate included in life signals, and may transmit an alarm about an emergency to the user terminal 170 based on the renewed determination on whether the user is in the space when it is continuously determined that the user is in the space for a longer time than a predetermined fourth period of time. Once an emergency is sensed, the emergency sensing unit 430 may deliver information about the emergency to the artificial intelligence speaker 130 to confirm whether the emergency has actually occurred based on a user's response, thereby preventing errors in sensing an emergency. Once it is confirmed that the emergency has occurred based on the interaction between the artificial intelligence speaker 130 and the user, the emergency sensing unit 430 may deliver information about the emergency to the control system and the user terminal 170 to perform an operation for emergency measures.

For example, when there are not a user's continuous movements for three seconds or more, the emergency sensing unit 430 may collect data on the user's heart rate and breathing. When the data on the user's breathing and heart rate are collected, it may be determined that the user is in the space even though the user's movements are not sensed. In addition, when it is continuously determined that the user is in the space for three hours or more, the emergency sensing unit 430 may provide an alarm about the occurrence of an emergency. Here, the emergency sensing unit 430 may not determine that an emergency has occurred when determining that the user is sleeping in consideration of the time range.

According to an embodiment of the present disclosure, the emergency sensing unit 430 may determine whether each of a user's respiratory rate, heart rate, and body temperature is out of the normal range, which has been preset, and may generate an alarm when the measured number of times of each item exceeds the reference number of times. In other words, as a basis for transmitting the event value in the event of an emergency, the emergency sensing unit 430 may monitor whether the value of each of the user's respiratory rate, heart rate, and body temperature falls below or above the normal ranges thereof. The emergency sensing unit 430 may monitor the heart rate, the respiratory rate, and the body temperature for three or five minutes because the user could move in the middle of measuring them, and may measure how many times the heart rate and the respiratory rate exceed the normal range and monitor a change in the body temperature.

For example, because biometric data is delivered every one second or three seconds, an alarm about an emergency may be sent whenever a user's heart rate or breathing rate exceeds the normal range 15, 30, or 60 times or more for three or five minutes.

According to an embodiment of the present disclosure, the emergency sensing unit 430 may set the normal range (GREEN ZONE), the boundary range (BLUE ZONE), and the emergency range (Red Zone) for each of a user's respiratory rate and heart rate, may collect the distribution in the boundary range and the emergency range whenever the respiratory rate and the heart rate exceed the normal range, may determine the grade of an emergency based on the pattern and the ratio for each range of distribution, and may differentially generate alarms based on the grades.

More specifically, the normal range, the boundary range, and the emergency range for a respiratory rate and a heart rate may be set as shown in Table 2 below.

TABLE 2

| | RED | BLUE | | |
|---|---|---|---|---|
| | ZONE | ZONE | GREEN ZONE | |
| HEART RATE | 50 or less | 51~59 | 60~100 | 101~110 |
| (TIMES/MINUTE) | 150 or more | 111~149 | | |
| RESPIRATORY RATE | 8 or less | 9 | 10~14 | 15~24 |
| (TIMES/MINUTE) | 30 or more | 25~29 | | |

In addition, the emergency sensing unit 430 may set the normal range, the boundary range, and the emergency range for each of a respiratory rate and a heart rate as shown in Table 2 above, and, may collect the accumulated distribution in the boundary range and the emergency range when the respiratory rate and the heart rate exceed the normal range. The emergency sensing unit 430 may independently monitor the distribution in the boundary range and the emergency range and calculate the pattern and the ratio of the distribution for each range. The emergency sensing unit 430 may grade an emergency based on the pattern and the ratio of the distribution for each range. For example, depending on the severity of the emergency, emergencies may be rated as high, medium, and low, not limited thereto, and may be subdivided into various levels.

According to an embodiment of the present disclosure, the emergency sensing unit 430 may build a learning model that grades emergencies by learning information about the features of the pattern and the ratio of the distribution for each range. More specifically, the emergency sensing unit 430 may generate a feature map for the pattern of the distribution for each range and extract a feature vector as feature information from the feature map. In addition, the emergency sensing unit 430 may generate a feature vector regarding the ratio of the distribution for each range, and may construct a learning model by learning feature vectors regarding the pattern and the ratio of the distribution for each range as learning data.

Feature maps for the pattern of the distribution for each range may be independently used, and, if necessary, they may be integrated into one feature map. The learning model may receive information about the features of the pattern and the ratio of the distribution for each range as input and generate the results of grading emergencies as output. Therefore, it may be possible for the emergency sensing unit 430 to efficiently grade an emergency based on information obtained by monitoring a respiratory rate, a heart rate, and a body temperature through a learning model, when the occurrence of the emergency is sensed.

According to an embodiment of the present disclosure, the emergency sensing unit 430 may determine the status of a user's body temperature based on the result of measuring the user's body temperature, which is sent each time the user having the habit of measuring body temperature measures his/her body temperature or the user's body temperature is measured by a self-measurement. Here, the emergency sensing unit 430 may classify the state of the user's body temperature into normal, mild fever, and high fever based on information on the state of each section of temperature received from the radar sensor 110 as a result of measuring the user's body temperature. In addition, when the state of the user's body temperature is classified as high fever, the emergency sensing unit 430 may enable the artificial intelligence speaker 130 to output a voice message to re-measure the user's body temperature after a certain period of time (e.g., three hours later). The emergency sensing unit 430 may prevent the voice message from being output when the time at which the voice message for re-measuring the user's body temperature is to be output falls within the set time zone (e.g., after 19:00).

When the emergency call response unit 450 receives an emergency call event signal from the life signal receiver 410, it may provide an alarm about a user's request for an emergency call to a pre-designated manager or guardian. For example, the emergency call response unit 450 may provide information about the user's situation to a pre-designated manager or guardian by sending an emergency text message thereto.

The control unit 470 may control the overall operation of the monitoring server 150 and manage control flow or data flow between the life signal receiver 410, the emergency sensing unit 430, and the emergency call response unit 450.

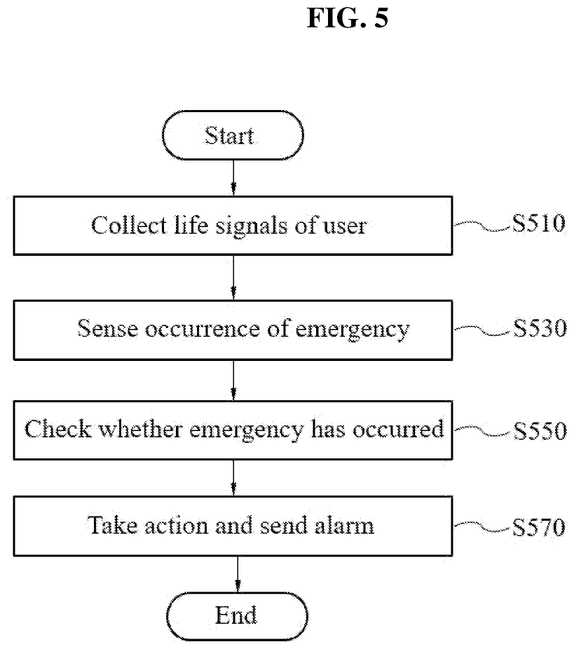

FIG. 5 is a flowchart for illustrating an embodiment of the operation of the system for providing care services according to the present disclosure.

Referring to FIG. 5, the system 100 for providing care services may connect the radar sensor 110 and the artificial intelligence speaker 130 to allow a user to get into the habit of measuring his/her body temperature and provide care services by monitoring emergency situations.

The system 100 for providing care services may collect a user's life signals through the radar sensor 110 installed in a specific space at S510. That is, the radar sensor 110 may sense the life signals of the user inside the specific space, and the monitoring server 150 may receive the life signals from the radar sensor 110. The life signals may include information on whether the user is in the space, whether the user is moving, the user's breathing rate, the user's heart rate, and the user's body temperature. For example, the radar sensor 110 may be installed on a wall or a table in the space, not necessarily limited thereto, and may be installed on various positions such as the ceiling depending on the conditions of the place. The radar sensor 110 may set its measuring angle by adjusting its direction depending on where it is installed, and may transmit a radar signal into a sensing area formed in front and then receive a reflected signal to determine whether a user is in the space, whether the user is moving, the user's breathing rate, the user's heart rate, and the user's body temperature. The radar sensor 110 may transmit the measured life signals to the monitoring server 150 based on a predefined communication protocol.

Afterwards, the system 100 for providing care services may analyze the life signals through the monitoring server 150 and may sense the occurrence of an emergency situation based on the result of the analysis at S530. When an emergency situation is sensed, the system 100 for providing care services may output a voice message through the artificial intelligence speaker 130 to determine whether the emergency situation has actually occurred based on the user's response at S550.

Meanwhile, when it is confirmed that the emergency situation has occurred, the system 100 for providing care services may quickly send an alarm about the situation and take action in response to the situation at S570. For example, the monitoring server 150 may send an alerting text message regarding the emergency situation to a pre-designated manager or guardian, and may deliver information about the situation to an external system such as a pre-designated rescue organization or control center so that action is taken quickly in response to the situation.

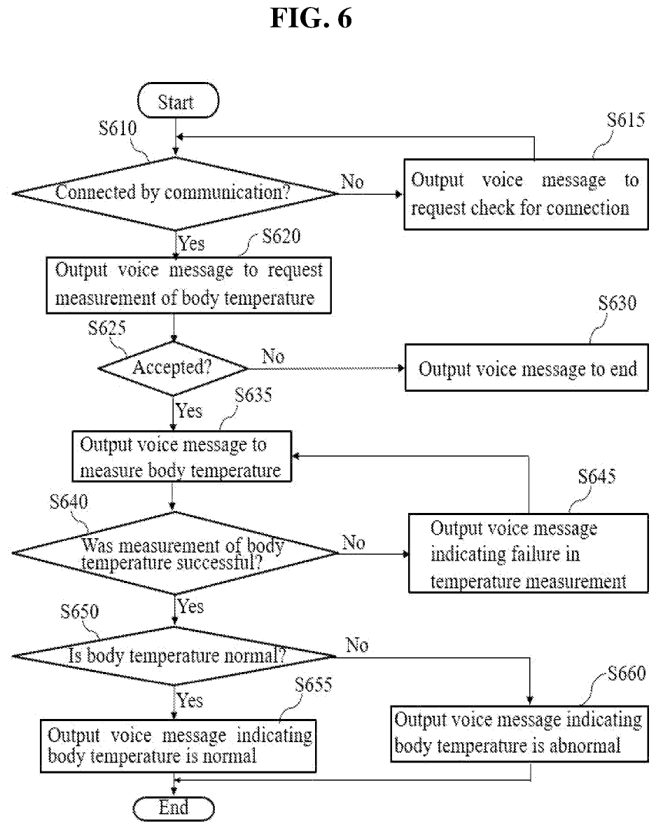

FIG. 6 is a flowchart for illustrating the process of measuring body temperature based on voice messages automatically output by the artificial intelligence speaker of the system for providing care services according to the present disclosure.

Referring to FIG. 6, the artificial intelligence speaker 130 may determine whether it is connected by communication with the radar sensor 110 at S610. When not connected by communication with the radar sensor 110, the artificial intelligence speaker 130 may output a voice message to request a check for the connection at S615. For example, the artificial intelligence speaker 130 may automatically output a voice message: “The body temperature sensor is not connected. Please check whether it is connected. If there is a problem, please contact the person in charge.” When connected with the radar sensor 110 by communication, the artificial intelligence speaker 130 may output a voice message to make a request for measuring body temperature at S620. For example, to encourage a user to measure his/her body temperature, the artificial intelligence speaker 130 may automatically output a voice message: “Try measuring your body temperature today for your health. If you want to measure your temperature, please say ‘yes.’ If you do not want to measure your temperature, please say ‘no’.” The artificial intelligence speaker 130 may automatically output a voice message to encourage the user to measure his/her body temperature at a designated time. For example, the artificial intelligence speaker 130 may automatically output a voice message to make a request for measuring body temperature every day at 10 o'clock in order to allow the user to make a habit of measuring his/her body temperature every day at the time.

The artificial intelligence speaker 130 may recognize the user's voice response to the voice message to make a request for measuring body temperature and determine whether the user will measure the temperature at S625. When the user does not want to measure his/her body temperature, the artificial intelligence speaker 130 may output a voice message indicating the end of the process at S630 and then end the process. For example, when the user says “no,” the artificial intelligence speaker 130 may end the process of measuring body temperature after automatically outputting a voice message: “Yes, I hope you live a healthy life by measuring your body temperature regularly. Have a nice day.” When the user wants to measure his/her body temperature, the artificial intelligence speaker 130 may output voice messages to encourage the user to follow the procedure for measuring body temperature at S635. For example, when a user says “yes,” according to the order of measuring body temperature, the artificial intelligence speaker 130 may automatically output a voice message saying, “Please place your palms slightly away from the body temperature sensor and wait a moment while it counts,” and may wait five seconds for the user to follow the direction and then automatically output a voice message saying, “Your body temperature is being measured. Please do not lift your palms while counting to five. One, two, three, four, five. The temperature measurement has been completed.” As soon as the user's body temperature is measured by the body temperature sensor, the radar sensor 110 may send the value of the measured body temperature to the monitoring server 150.

The artificial intelligence speaker 130 may determine whether the measurement of body temperature was successful at S640. When a user's body temperature is not measured by the body temperature sensor or the value of the measured body temperature is below the measurement threshold (e.g., 34.9° C. or below), after outputting a voice message indicating that the measurement of body temperature has failed at S645, the artificial intelligence speaker 130 again may perform a series of steps starting from S630 to re-measure the user's body temperature. When the re-measurement of the user's body temperature also fails, the artificial intelligence speaker 130 may allow the body temperature to be measured at the user's request. When the measurement of the user's body temperature is successful, the artificial intelligence speaker 130 may determine whether the value of the body temperature is normal at S650. When the value of the measured body temperature is normal, the artificial intelligence speaker 130 may automatically output a voice message indicating that the body temperature is normal at S655. For example, the artificial intelligence speaker 130 may automatically output a voice message: “OOO, your body temperature is currently normal. Have a nice day.” When the value of the measured body temperature is not normal, the artificial intelligence speaker 130 may generate an alarm indicating that the body temperature is abnormal and automatically output a voice message to encourage the user to take some additional actions in response to the situation at S660. For example, the artificial intelligence speaker 130 may automatically output a voice message to recommend to go to a public health center or hospital in order to guide the user on what measures can be taken for the abnormal signs.

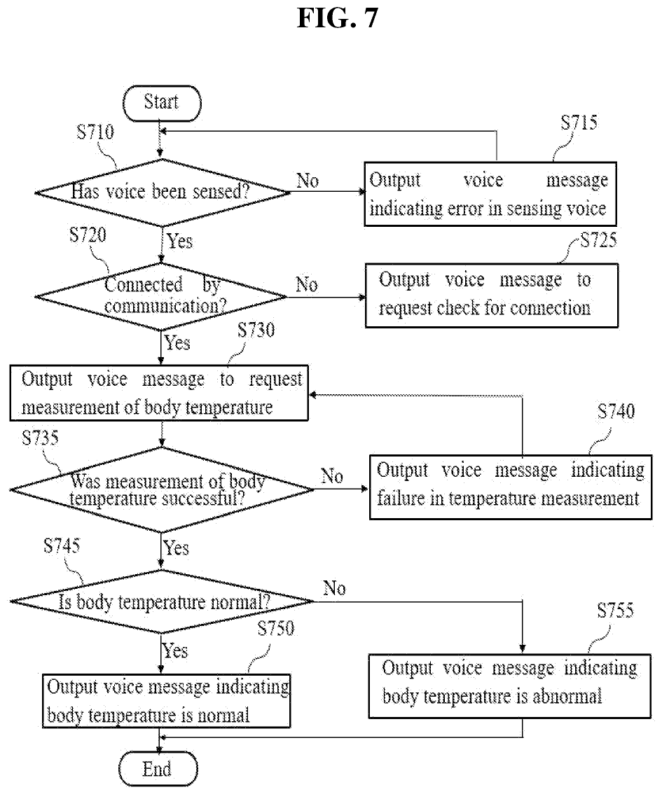

FIG. 7 is a flowchart for illustrating the process of measuring body temperature based on a user's voice messages by the system for providing care services according to the present disclosure.

Referring to FIG. 7, the artificial intelligence speaker 130 may determine whether a user's voice to make a request for measuring his/her body temperature has been sensed at S710. When such a voice has not been sensed, the artificial intelligence speaker 130 may output a usual message indicating that there is an error in sensing at S715. When a voice to make a request for measuring body temperature is sensed, the artificial intelligence speaker 130 may determine whether it is connected by communication with the radar sensor 110 at S720. When not connected by communication with the radar sensor 110, the artificial intelligence speaker 130 may output a voice message to request a check for the connection at S725. For example, the artificial intelligence speaker 130 may automatically output a voice message: “The body temperature sensor is not connected. Please check whether it is connected. If there is a problem, please contact the person in charge.” When connected with the radar sensor 110 by communication, the artificial intelligence speaker 130 may output voice messages to encourage the user to take the steps to measure his/her body temperature according to the order of measuring body temperature at S730. For example, according to the order of measuring body temperature, the artificial intelligence speaker 130 may automatically output a voice message saying, “Please place your palms slightly away from the body temperature sensor and wait a moment while it counts,” and may wait five seconds for the user to follow the direction and then automatically output a voice message saying, “Your body temperature is being measured. Please do not lift your palms while counting to five. One, two, three, four, five. The temperature measurement has been completed.” As soon as the user's body temperature is measured by the body temperature sensor, the radar sensor 110 may send the value of the measured body temperature to the monitoring server 150.

The artificial intelligence speaker 130 may determine whether the measurement of body temperature was successful at S735. When a user's body temperature is not measured by the body temperature sensor or the value of the measured body temperature is below the measurement threshold (e.g., 34.9° C. or below), after outputting a voice message indicating that the measurement of body temperature has failed at S740, the artificial intelligence speaker 130 again may perform a series of steps starting from S730 to re-measure the user's body temperature. When the re-measurement of the user's body temperature also fails, the artificial intelligence speaker 130 may allow the body temperature to be measured at the user's re-request. When the measurement of the user's body temperature is successful, the artificial intelligence speaker 130 may determine whether the value of the body temperature is normal at S745. When the value of the measured body temperature is normal, the artificial intelligence speaker 130 may automatically output a voice message indicating that the body temperature is normal at S750. For example, the artificial intelligence speaker 130 may automatically output a voice message: “OOO, your body temperature is currently normal. Have a nice day.” When the value of the measured body temperature is not normal, the artificial intelligence speaker 130 may generate an alarm indicating that the body temperature is abnormal and automatically output a voice message to encourage the user to take some additional actions in response to the situation at S755. For example, the artificial intelligence speaker 130 may automatically output a voice message to recommend to go to a public health center or hospital in order to guide the user on what measures can be taken for the abnormal signs.

The AI-based system for providing care services according to an embodiment of the present disclosure may enable elderly users to request rescue on their own and make a habit of measuring their body temperature. In addition, it may be possible for the system to monitor the users in real time to sense abnormal signs displayed by them and quickly respond thereto and to provide the users with care tailored to the abnormal signs.

Although the present disclosure has been described with reference to the desirable embodiments thereof herein, a person having ordinary skill in the art would understand that various modifications and changes can be made to the present disclosure within the technology and the scope of the present disclosure as set forth in the following claims.

What is claimed is:

1. An AI-based system for providing care services, comprising:
a radar sensor that is installed in a specific space, obtains, in the specific space, life signals including information on whether the user is in the space, the user's movements, the user's breathing, and the user's heart rate without being in contact with a user, and obtains information about the user's body temperature among the life signals by being in contact with the user;
an artificial intelligence speaker that is linked to the radar sensor, outputs a voice message to the user based on the information obtained by the radar sensor to determine whether an emergency has occurred to the user, and outputs a voice message to encourage the user to measure his/her body temperature at a specified time to allow the user to get into the habit of measuring his/her body temperature;
a monitoring server that receives the life signals collected by the radar sensor and analyzes the received life signals to monitor the specific space, senses an emergency that has occurred to the user and checks whether the emergency has actually occurred through the artificial intelligence speaker, and then provides an alarm about the emergency; and
a user terminal linked to each of the radar sensor, the artificial intelligence speaker, and the monitoring server through a dedicated application.

2. The system of claim 1, wherein the radar sensor includes:
a life sensing module that transmits a radar signal into a sensor sensing area formed in front and then receives a reflected signal to measure the life signals;
a body temperature measurement module that includes a body temperature sensor and measures the user's body temperature when in contact with the user through the body temperature sensor; and
a communication module that is connected, through a network, with the artificial intelligence speaker, the monitoring server, or the user terminal to provide a communication function.

3. The system of claim 1, wherein the artificial intelligence speaker includes:
a voice recognition module that determines whether an emergency has occurred by recognizing the user's voice;
an action alarm module that automatically outputs a voice message to encourage the user to measure his/her body temperature at a specified time, analyzes the user's voice response to determine whether the user wants to measure his/her body temperature, and outputs voice messages to encourage the user to take the steps to measure his/her body temperature according to the order of measuring body temperature when the user wants to measure his/her body temperature; and
a communication module that is connected, through a network, with the radar sensor, the monitoring server, or the user terminal to provide a communication function.

4. The system of claim 3, wherein the artificial intelligence speaker receives information collected by the radar sensor from the voice recognition module to carry out voice commands and generates an event signal when an obtained voice command corresponds to an emergency call.

5. The system of claim 3, wherein, when the user's voice message to proceed a self-measurement of his/her body temperature is sensed before the action alarm module automatically outputs a voice message to encourage the user to measure his/her body temperature at a specified time, the artificial intelligence speaker prevents the voice message to encourage the user to measure his/her body temperature from being automatically output.

6. The system of claim 4, wherein, when the event signal is received, the monitoring server sends an alarm about the user's emergency call to the user terminal of a pre-designated manager or guardian.

7. The system of claim 6, wherein, when the occurrence of an event resulting from the emergency call is sensed, the monitoring server provides biometric information about the user's breathing, the user's heart rate, and the user's body temperature along with an alarm about the occurrence of the event to an external designated organization.

8. The system of claim 1, wherein the monitoring server determines whether the user is in the space based on the life signals, monitors whether the user is moving based on whether the user is in the space, classifies the ranges of body temperature into unmeasurable, to be remeasured, normal, normal (slight fever), abnormal (high fever), and abnormal (high temperature and fire) and sets an alarm for each range, and sends an alarm to the user terminal of a pre-designated manger or guardian when the value of the measured body temperature falls within the range of abnormal (high fever) and allows the artificial intelligence speaker to output a voice message to re-measure the user's body temperature after a certain period of time.
